(19) United States
(12) Patent Application Publication
(10) Pub. No.: US 2009/0181932 A1
(43) Pub. Date: Jul. 16, 2009

(54) COMPOSITIONS CONTAINING O-SULFATE AND O-PHOSPHATE CONTAINING ARYL SULFONAMIDE DERIVATIVES USEFUL AS beta-AMYLOID INHIBITORS

(75) Inventors: David Zenan LI, Princeton, NJ (US); Dane Springer, Yardley, PA (US); Sayed Elmarakby, Newtown, PA (US); Appavu Chandrasekaran, Plainsboro, NJ (US); Anthony F. Kreft, III, Langhorne, PA (US)

Correspondence Address: HOWSON & HOWSON LLP / WYETH, 501 OFFICE CENTER DRIVE, SUITE 210, FORT WASHINGTON, PA, 19034, US

(73) Assignee: Wyeth, Madison, NJ (US)

(21) Appl. No.: 12/349,850

(22) Filed: Jan. 7, 2009

Related U.S. Application Data
(60) Provisional application No. 61/010,894, filed on Jan. 11, 2008.

(51) Int. Cl. A61K 31/18; C07D 333/34; C07C 305/02; A61K 31/66; A61P 35/00; A61P 25/28; C07F 9/09; A61K 31/381

(52) U.S. Cl. 514/117; 549/65; 558/29; 558/22; 514/445; 514/604

(57) ABSTRACT

A synthetic compound characterized by having the structure of formula I or II or a pharmaceutically acceptable salt and/or hydrate thereof is provided. Formula I and formula II are defined as follows.

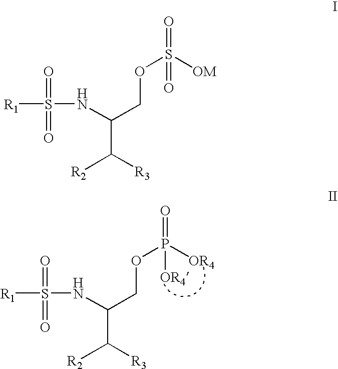

wherein R₁ is substituted aryl or substituted heteroaryl; R₂ and R₃ are independently selected from the group consisting of CF₃, substituted phenyl, C₁-C₄ alkyl, substituted C₁-C₄ alkyl, (CF₃)ₙC₁-C₄alkyl, (CF₃)ₙ(substituted C₁-C₄ alkyl), provided that when R₂ or R₃ is CF₃, the other is not an unsubstituted alkyl; R₄ and R_{4′} are independently selected from the group consisting of M, C₁-C₄ alkyl, phenyl, and benzyl, wherein M is a metal ion is selected from the group consisting of sodium, lithium, calcium, magnesium and potassium, or R₄ and R_{4′} are taken together to form a cyclic structure. Methods of making such compounds and uses thereof are also provided.

### CROSS-REFERENCE TO RELATED APPLICATION

This application claims the benefit under 35 USC 119(e) of prior U.S. Provisional Patent Application No. 61/010,894, filed Jan. 11, 2008.

### BACKGROUND OF THE INVENTION

This invention relates to inhibitors of beta amyloid production, which have utility in the treatment of Alzheimer's disease and/or cancer.

The β-amyloid precursor protein (APP) and the Notch receptor undergo intramembranous proteolysis by the Presenilin-dependent γ-secretase. The cleavage of APP by γ-secretase releases amyloid-β peptides, which have been implicated in the pathogenesis of Alzheimer's disease as well as a variety of other conditions, and the APP intracellular domain. A similar γ-secretase-mediated cleavage of the Notch receptor liberates the Notch intracellular domain (NICD). NICD translocates to the nucleus and activates the transcription of genes that regulate the generation, differentiation, and survival of neuronal cells. Hence, some of the effects of APP signaling and Alzheimer's disease (AD) have been described as being mediated by the interaction of APP and Notch.

To date, several small molecule drugs have been described as inhibiting beta amyloid production. Such drugs include heterocyclic sulfonamide compounds such as those described in U.S. Pat. No. 6,878,742 and U.S. Pat. No. 6,610,734 and fluoro- and trifluoroalkyl-containing heterocyclic sulfonamide compounds such as those described in U.S. Pat. No. 7,300,951 and US-2007-0254929-A1. Still other drugs include the phenylsulfonamides described in U.S. Pat. No. 7,166,622, or the drugs described in US-2005-0171180.

More recently, the use of gamma secretase inhibitors (GSIs) has been described as being useful to treat glaucoma by virtue of reducing antibody production. For example, see Proc Natl Acad Sci, 104 1344 (2007). GSIs may affect Notch processing, which is useful in treatment of certain forms of cancer.

What are needed in the art are alternate compounds useful for inhibiting β-amyloid, modulating Notch activity, and/or other indications.

### SUMMARY OF THE INVENTION

The invention describes a series of O-sulfate-containing aryl or heteroarylsulfonamide derivatives, and O-phosphate derivatives of formula (I) and formula (II) provided below. These formulae include prodrugs which permit alternative administration routes and are designed to provide better pharmacokinetic profiles, e.g., absorption, bioavailability and longer half-life.

In one embodiment, these compounds are formulated in pharmaceutical compositions useful as inhibitors of beta amyloid protein production from APP.

In another embodiment, methods of using a compound and/or composition provided herein for the treatment of physiological conditions due to increased beta amyloid levels (e.g., AD, Down's Syndrome) are provided.

In still another embodiment, these compounds are formulated in pharmaceutical compositions useful in treatment of cancers associated with notch processing. A method of treating such cancers using these compounds is provided.

In a further aspect, pharmaceutical kits are described. The kits have a container which includes a pharmaceutical composition described herein.

In yet another aspect, methods are described for preparing a compound of formula (I) or formula (II).

Still other used and advantages of the invention will be readily apparent from the following detailed description of the invention.

### DETAILED DESCRIPTION OF THE INVENTION

Pharmaceutical formulations containing a compound of formula I or II and their use are provided. Such compounds are useful in inhibiting beta amyloid production in patients susceptible to, or suffering from, AD or other diseases resulting from elevated levels of beta amyloid protein in the brain. The compounds of formula I and II include pharmaceutically acceptable salts and/or hydrates thereof, wherein:

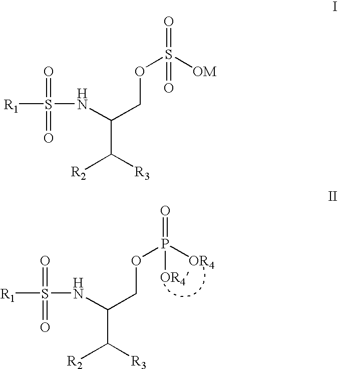

R₁ is substituted aryl or substituted heteroaryl;

R₂ and R₃ are independently selected from the group consisting of CF₃, substituted phenyl, C₁-C₄ alkyl, substituted C₁-C₄ alkyl, (CF₃)ₙC₁-C₄ alkyl, (CF₃)ₙ(substituted C₁-C₄ alkyl), wherein n is 1-3; provided that when R₂ or R₃ is CF₃, the other is not an unsubstituted alkyl; and

R₄ and R_{4′} are independently selected from the group consisting of M, C₁-C₄ alkyl, phenyl, and benzyl, wherein M is a metal ion chosen from sodium, lithium, calcium, magnesium and potassium; Or

R₄ and R_{4′} are taken together to form a cyclic phosphonate structure derived from a C₂₋₃ α,ω diol structure or a catechol derivative. In one embodiment, the cyclic structure is selected from the group consisting of:

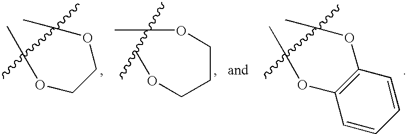

In formula II, the dotted semi-circle represents the option for R₄ and R_{4′} to be independent as defined herein or joined together to form a cyclic structure as described.

In one embodiment, R₁ is a substituted phenyl, a substituted thiophene, or a substituted pyridyl, which forms a substituted phenylsulfonamide, substituted thiophenesulfonamide, or a substituted pyridylsulfonamide, respectively, with S-stereochemistry at the carbon bearing the sulfonamide nitrogen atom.

The pharmaceutically acceptable salts are those derived from such organic and inorganic bases such as sodium hydroxide, sodium bicarbonate, potassium hydroxide, calcium hydroxide, magnesium hydroxide, lithium hydroxide, diethanolamine, ethylenediamine and similarly known acceptable bases.

The term “alkyl” is used herein to refer to both straight- and branched-chain saturated aliphatic hydrocarbon groups. In one embodiment, an alkyl group has 1 to about 8 carbon atoms (i.e., C₁, C₂, C₃, C₄, C₅ C₆, C₇, or C₈). In another embodiment, an alkyl group has 1 to about 6 carbon atoms (i.e., C₁, C₂, C₃, C₄, C₅ or C₆). In a further embodiment, an alkyl group has 1 to about 4 carbon atoms (i.e., C₁, C₂, C₃, or C₄).

The term “cycloalkyl” is used herein to refer to cyclic, saturated aliphatic hydrocarbon groups. In one embodiment, a cycloalkyl group has 3 to about 8 carbon atoms (i.e., C₃, C₄, C₅, C₆, C₇, or C₈). In another embodiment, a cycloalkyl group has 3 to about 6 carbon atoms (i.e., C₃, C₄, C₅ or C₆).

The terms “substituted alkyl” and “substituted cycloalkyl” refer to alkyl and cycloalkyl groups, respectively, having one or more substituents including, without limitation, hydrogen, halogen, CN, OH, NO₂, amino, aryl, heterocyclic, alkoxy, aryloxy, alkylcarbonyl, alkylcarboxy, alkylamino, and arylthio.

The term “arylthio” as used herein refers to the S(aryl) group, where the point of attachment is through the sulfur-atom and the aryl group can be substituted as noted herein.

The term “alkoxy” as used herein refers to the O(alkyl) group, where the point of attachment is through the oxygen-atom and the alkyl group can be substituted as noted herein.

The term “aryloxy” as used herein refers to the O(aryl) group, where the point of attachment is through the oxygen-atom and the aryl group can be substituted as noted herein.

The term “alkylcarbonyl” as used herein refers to the C(O)(alkyl) group, where the point of attachment is through the carbon-atom of the carbonyl moiety and the alkyl group can be substituted as noted herein.

The term “alkylcarboxy” as used herein refers to the C(O)O(alkyl) group, where the point of attachment is through the carbon-atom of the carboxy moiety and the alkyl group can be substituted as noted herein.

The term “alkylamino” as used herein refers to both secondary and tertiary amines where the point of attachment is through the nitrogen-atom and the alkyl groups can be substituted as noted herein. The alkyl groups can be the same or different.

The term “halogen” as used herein refers to Cl, Br, F, or I groups.

The term “aryl” as used herein refers to an aromatic, carbocyclic system, e.g., of about 6 to 14 carbon atoms, which can include a single ring or multiple aromatic rings fused or linked together where at least one part of the fused or linked rings forms the conjugated aromatic system. The aryl groups include, but are not limited to, phenyl, naphthyl, biphenyl, anthryl, tetrahydronaphthyl, phenanthryl, indene, benzonaphthyl, and fluorenyl.

The term “substituted aryl” refers to an aryl group which is substituted with one or more substituents including halogen, CN, OH, NO₂, amino, alkyl, cycloalkyl, alkenyl, alkynyl, C₁ to C₃ perfluoroalkyl, C₁ to C₃ perfluoroalkoxy, aryloxy, alkyloxy including —O—(C₁ to C₁₀ alkyl) or —O—(C₁ to C₁₀ substituted alkyl), alkylcarbonyl including —CO—(C₁ to C₁₀ alkyl) or —CO—(C₁ to C₁₀ substituted alkyl), alkylcarboxy including —COO—(C₁ to C₁₀ alkyl) or —COO—(C₁ to C₁₀ substituted alkyl), —C(NH₂)═N—OH, —SO₂—(C₁ to C₁₀ alkyl), —SO₂—(C₁ to C₁₀ substituted alkyl), —O—CH₂-aryl. Also included within suitable substituents are alkylamino, arylthio, aryl, and heteroaryl, which groups can be themselves substituted. Desirably, a substituted aryl group is substituted with 1 to about 4 substituents. The term, “alkenyl” refers to both straight- and branched-chain alkyl groups with at least one carbon-carbon double bond and two to eight carbon atoms, preferably two to six carbon atoms. An “alkynyl” group is intended to cover both straight- and branched-chain alkyl groups with at least one carbon-carbon triple bond and two to eight carbon atoms, preferably two to six carbon atoms.

The term “heterocycle” or “heterocyclic” as used herein can be used interchangeably to refer to a stable, saturated or partially unsaturated 3- to 9-membered monocyclic or multicyclic heterocyclic ring. The heterocyclic ring has in its backbone carbon atoms and one or more heteroatoms including nitrogen, oxygen, and sulfur atoms. In one embodiment, the heterocyclic ring has 1 to about 4 heteroatoms in the backbone of the ring. When the heterocyclic ring contains nitrogen or sulfur atoms in the backbone of the ring, the nitrogen or sulfur atoms can be oxidized. The term “heterocycle” or “heterocyclic” also refers to multicyclic rings in which a heterocyclic ring is fused to an aryl ring of about 6 to about 14 carbon atoms. The heterocyclic ring can be attached to the aryl ring through a heteroatom or carbon atom provided the resultant heterocyclic ring structure is chemically stable. In one embodiment, the heterocyclic ring includes multicyclic systems having 2 to 5 rings.

A variety of heterocyclic groups are known in the art and include, without limitation, oxygen-containing rings, nitrogen-containing rings, sulfur-containing rings, mixed heteroatom-containing rings, fused heteroatom containing rings, and combinations thereof. Examples of heterocyclic groups include, without limitation, tetrahydrofuranyl, piperidinyl, 2-oxopiperidinyl, pyrrolidinyl, morpholinyl, thiamorpholinyl, thiamorpholinyl sulfoxide, pyranyl, pyronyl, dioxinyl, piperazinyl, dithiolyl, oxathiolyl, dioxazolyl, oxathiazolyl, oxazinyl, oxathiazinyl, benzopyranyl, benzoxazinyl and xanthenyl.

The term “heteroaryl” as used herein refers to a stable, aromatic 5- to 14-membered monocyclic or multicyclic heteroatom-containing ring. The heteroaryl ring has in its backbone carbon atoms and one or more heteroatoms including nitrogen, oxygen, and sulfur atoms. In one embodiment, the heteroaryl ring contains 1 to about 4 heteroatoms in the backbone of the ring. When the heteroaryl ring contains nitrogen or sulfur atoms in the backbone of the ring, the nitrogen or sulfur atoms can be oxidized. The term “heteroaryl” also refers to multicyclic rings in which a heteroaryl ring is fused to an aryl ring. The heteroaryl ring can be attached to the aryl ring through a heteroatom or carbon atom provided the resultant heterocyclic ring structure is chemically stable. In one embodiment, the heteroaryl ring includes multicyclic systems having 2 to 5 rings.

A variety of heteroaryl groups are known in the art and include, without limitation, oxygen-containing rings, nitrogen-containing rings, sulfur-containing rings, mixed heteroatom-containing rings, fused heteroatom containing rings, and combinations thereof. Examples of heteroaryl groups include, without limitation, furyl, pyrrolyl, pyrazolyl, imidazolyl, triazolyl, pyridyl, pyridazinyl, pyrimidinyl, pyrazinyl, triazinyl, azepinyl, thienyl, dithiolyl, oxathiolyl, oxazolyl, thiazolyl, oxadiazolyl, oxatriazolyl, oxepinyl, thiepinyl, diazepinyl, benzofuranyl, indolyl, benzazolyl, purindinyl, pyranopyrrolyl, isoindazolyl, indoxazinyl, benzoxazolyl, quinolinyl, isoquinolinyl, benzodiazonyl, napthylridinyl, benzothienyl, pyridopyridinyl, acridinyl, carbazolyl, and purinyl rings.

The term “substituted heterocycle” and “substituted heteroaryl” as used herein refers to a heterocycle or heteroaryl group having one or more substituents including halogen, CN, OH, NO₂, amino, alkyl, cycloalkyl, alkenyl, alkynyl, C₁ to C₃ perfluoroalkyl, C₁ to C₃ perfluoroalkoxy, aryloxy, alkyloxy including —O—(C₁ to C₁₀ alkyl) or —O—(C₁ to C₁₀ substituted alkyl), alkylcarbonyl including —CO—(C₁ to C₁₀ alkyl) or —CO—(C₁ to C₁₀ substituted alkyl), alkylcarboxy including —COO—(C₁ to C₁₀ alkyl) or —COO—(C₁ to C₁₀ substituted alkyl), —C(NH₂)═N—OH, —SO₂—(C₁ to C₁₀ alkyl), —SO₂—(C₁ to C₁₀ substituted alkyl), —O—CH₂-aryl, alkylamino, arylthio, aryl, aryl, or heteroaryl, which groups may be optionally substituted. A substituted heterocycle or heteroaryl group may have 1, 2, 3, or 4 substituents.

It has been found by the present inventors that at least one of the compounds of the invention is generated in vivo as a metabolite of a compound described as a beta amyloid inhibitor. U.S. Pat. No. 6,610,734 and U.S. Pat. No. 7,300,951. Unexpectedly, however, when the “metabolite” was prepared synthetically and administered, it was found to function as a prodrug. It is believed that the compound is metabolized differently following parenteral administration, thereby improving bioavailability. Thus, the compounds of formula (I) and (II) are anticipated to also be less metabolized by a variety of metabolic pathways following parenteral administration, in particular glucuronidation of the 2-OH of the 1,2 amino alcohol of parent compounds III (see Scheme I) to yield 2-glucuronide metabolites. Therefore, these compounds are expected to increase bioavailability for efficient therapy and may limit side effects. The protonated or anionic sulfates and phosphates should have improved water solubility and allow for intravenous drug delivery. Still other uses and advantages of these compounds are provided herein.

In one embodiment, the compounds of the invention are free of biological materials with which they may be associated in vivo. This can be readily accomplished by producing the compounds synthetically. Alternatively, the compounds are isolated or purified from biological materials.

### Synthesis

The compounds of the present invention can be prepared using the methods described below, together with synthetic methods known in the synthetic organic arts or variations of these methods by one skilled in the art. These methods include, but are not limited to, those outlined below.

The compounds of type I of this invention can be prepared according to the methods outlined in Scheme 1 below:

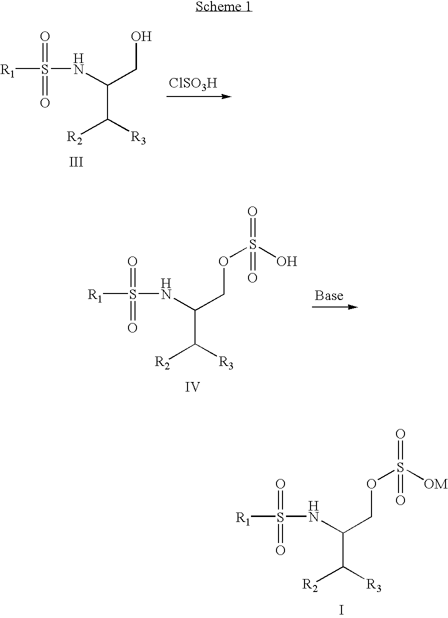

The sulfonamide alcohol of type III is treated with commercially available chlorosulfonic acid in a suitable cold solvent (e.g., at 0° C.), or other sulfonating agents known in the art, under an inert atmosphere such as nitrogen or argon to provide compounds of formula IV. Suitable methods for syntheses of alcohols of type III have been described in US 2004/198778 and US 2003/013892; International Patent Publication Nos. WO 2002/057252; WO 2003/050063; WO 2003/103660; and U.S. Patent Application No. 60/959,675, filed Jul. 16, 2007. Compounds of formula IV without further purification are treated with a base which serves as the source of the metal ion, M, to afford the sulfates of formula I. Suitable bases include, e.g., as sodium bicarbonate or other organic or inorganic bases. Recrystallization is performed using a combination of suitable solvents as needed.

Compounds of type II of this invention can be prepared according to methods well known to those skilled in the art. The most typical procedure, shown in Scheme 2, involves the reaction of a sulfonamide alcohol of type III with a chlorophosphonate derivative, or another phosphonating agent, in the presence of a suitable organic or inorganic base to afford phosphonates of type II. In one embodiment, the cyclic phosphate prodrug is the cycloSal moiety [C. Meier, Eur J Org Chem, 2006, 1081-1102]. These compounds can be simple alkyl, aryl, or cyclic derivatives that can be hydrolytically cleaved by phosphatases in vivo rendering the parent alcohols of type III.

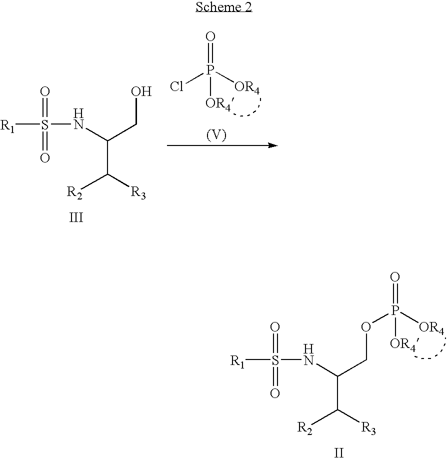

If the R₄ or R_{4′} group is a benzyl substituent, the compound can be deprotected via hydrogenation to afford the protonated form of the phosphonate II [L J Silverberg et al, Organic Process Res and Dev., 2000, 4:34-42].

The R₄ and/or the R_{4′} groups can be a 2,2,2-trichloroethyl group. Such a group can be removed via reductive methods using zinc-copper coupling or other methods known to those skilled in the art [G R Pettit, et al, Anti-cancer Drug Design, 1995, 10, 299-309]. The metal salt product of this reduction can be converted to anionic phosphate derivatives with an appropriate metal cation using ion-exchange methods.

### Methods of Using the Compounds

Compounds within formulae (I) and (II) are inhibitors of beta amyloid production and may further have notch activity. In preliminary studies using protease specific assays, exemplary compounds of formulae (I) and (II) have been shown to exhibit specific inhibition with respect to protease activity. Thus, the compounds are useful for treatment and prevention of a variety of conditions in which modulation of beta amyloid levels provides a therapeutic benefit. Such conditions include, e.g., amyloid angiopathy, cerebral amyloid angiopathy, systemic amyloidosis, Alzheimer's disease (AD), hereditary cerebral hemorrhage with amyloidosis of the Dutch type, inclusion body myositis, Down's syndrome, mild cognitive impairment (MCI), among others. Desirably, the compounds are administered in an amount sufficient to alleviate the symptoms and/or progress of the condition.

In one embodiment, a method of treating a neoplasm in a subject is provided. The method involves administering to a subject in need thereof a compound of the invention as the sole active agent or in a regimen involving one or more other active agent or other therapies (e.g., hormonal therapy, immunotherapy, anti-angiogenesis therapy, targeted therapy (e.g., therapy directed to a cancer target such as Gleevec® and other tyrosine kinase inhibitors, Velcade®, Sutent®), and radiation treatment). The neoplasm may be a hematologic cancer or a solid tumor.

In one embodiment, the solid tumor is fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, uterine cancer, testicular cancer, small cell lung carcinoma, bladder carcinoma, lung cancer, epithelial carcinoma, glioma, glioblastoma multiforme, astrocytoma, medulloblastoma, craniopharyngioma, ependymoma, pinealoma, hemangioblastoma, acoustic neuroma, oligodendroglioma, meningioma, skin cancer, melanoma, neuroblastoma, or retinoblastoma.

In addition, the compounds of formulae (I) and (II) may be utilized in generating reagents useful in diagnosis of conditions associated with abnormal levels of beta amyloid or with notch activity. For example, the compounds of formulae (I) and/or (II) may be used to generate antibodies, which would be useful in a variety of diagnostic assays. Methods for generating monoclonal, polyclonal, recombinant, and synthetic antibodies or fragments thereof, are well known to those of skill in the art. See, e.g., E. Mark and Padlin, “Humanization of Monoclonal Antibodies”, Chapter 4, The Handbook of Experimental Pharmacology, Vol. 113, The Pharmacology of Monoclonal Antibodies, Springer-Verlag (June, 1994); Kohler and Milstein and the many known modifications thereof; International Patent Publication No. WO 86/01533; British Patent Application Publication No. GB2188638A; Amit et al., Science, 233:747-753 (1986); Queen et al., Proc. Nat'l. Acad. Sci. USA, 86:10029-10033 (1989); International Patent Publication No. WO90/07861; and Riechmann et al., Nature, 332:323-327 (1988); Huse et al, Science, 246:1275-1281 (1988), which are hereby incorporated by reference. Alternatively, the compounds of formulae (I) and/or (II) may themselves be used in such diagnostic assays. Regardless of the reagent selected (e.g., antibody or compound of formula (I)), suitable diagnostic formats including, e.g., radioimmunoassays and enzyme-linked immunosorbent assays (ELISAs), are well known to those of skill in the art and are not a limitation.

Additionally, cellular, cell-free and in vivo screening methods to detect inhibitors of beta amyloid production are known in the art. Such assays may include radioimmunoassays and enzyme-linked immunosorbent assay (ELISA), among others. See, e.g., P. D. Mehta, et al., Techniques in Diagnostic Pathology, vol. 2, eds., Bullock et al, Academic Press, Boston, pages 99-112 (1991), International Patent Publication No. WO 98/22493, European Patent No. 0652009, U.S. Pat. No. 5,703,129 and U.S. Pat. No. 5,593,846, which are hereby incorporated by reference. Selection of an appropriate in vitro or in vivo screening assay is not a limitation.

In one embodiment, methods of inhibiting beta amyloid production in a subject are provided and include delivering a pharmaceutical composition containing a compound of formulae (I) and (II) to the subject.

In another embodiment, a method of increasing the circulating half-life of a compound useful in lowering beta amyloid levels in a subject by administering a compound having the structure of formula (I) or (II) or a pharmaceutically acceptable salt and/or hydrate thereof is provided. For example, the compound of formula (I) may be a prodrug of the compound 5-Chloro-thiophene-2-sulfonic acid [(1S,2R)-2-(3,5-difluoro-phenyl)-3,3,3-trifluoro-1-hydroxymethyl-propyl]-amide or of the compound 5′-chloro-N-[3,3,3-trifluoro-2-(trifluoromethyl)-1-S-(hydroxymethyl)propyl]thiophene-2′-sulfonamide. In one embodiment, 5-Chloro-thiophene-2-sulfonic acid [(1S,2R)-2-(3,5-difluoro-phenyl)-3,3,3-trifluoro-1-hydroxymethyl-propyl]-amide has the structure:

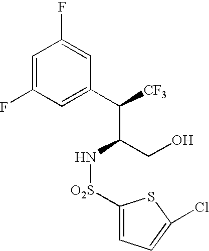

In one embodiment, 5′-chloro-N-[3,3,3-trifluoro-2-(trifluoromethyl)-1-S-(hydroxymethyl)propyl]thiophene-2′-sulfonamide has the structure:

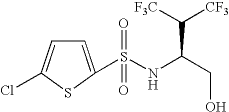

### Pharmaceutical Compositions and Formulations

Also provided are pharmaceutical compositions which contain one or more compounds of formula (I) or (II), or combinations thereof.

The compositions described herein may be administered to a subject by any desirable route, taking into consideration the specific condition for which it has been selected. By subject is meant any suitable human which has been recognized as having or at risk of having one or more of the conditions for which modulation of beta amyloid levels or Notch activity is desirable. Thus, the compositions containing compounds of formulae (I) and (II) are useful for treatment and/or prevention of a number of human conditions. As used herein, “prevention” encompasses prevention of symptoms in a subject who has been identified as at risk for the condition, but has not yet been diagnosed with the same and/or who has not yet presented any symptoms thereof.

These compounds may be delivered or administered by any suitable route of delivery, e.g. oral, injection, inhalation (including intranasal and intratracheal), transdermal, intravenous, subcutaneous, intramuscular, sublingual, intracranial, epidural, intratracheal, rectal, vaginal, among others. In one embodiment, the compositions are delivered for delivery of the compounds by a suitable parenteral route. In another embodiment, the compositions are formulated for intravenous delivery. The compounds are formulated in combination with conventional pharmaceutical carriers that are physiologically compatible. Optionally, one or more of the compounds of formulae (I) and (II) may be mixed with other active agents.

Suitable physiologically compatible carriers may be readily selected by one of skill in the art. For example, suitable solid carriers include, among others, one or more substances which may also act as flavoring agents, lubricants, solubilizers, suspending agents, fillers, glidants, compression aids, binders or tablet-disintegrating agents or an encapsulating material. In powders, the carrier is a finely divided solid, which is in admixture with the finely divided active ingredient. In tablets, the active ingredient is mixed with a carrier having the necessary compression properties in suitable proportions and compacted in the shape and size desired. The powders and tablets preferably contain up to 99% of the active ingredient. Suitable solid carriers include, for example, calcium or dicalcium phosphate, magnesium stearate, talc, starch, sugars (including, e.g., lactose and sucrose), cellulose (including, e.g., microcrystalline cellulose, methyl cellulose, sodium carboxymethyl cellulose), polyvinylpyrrolidine, low melting waxes, ion exchange resins, and kaolin.

Liquid carriers may be used in preparing solutions, suspensions, emulsions, syrups and elixirs. The active ingredient(s) can be dissolved or suspended in a pharmaceutically acceptable liquid carrier such as water, an organic solvent, a mixture of both or pharmaceutically acceptable oils or fat. The liquid carrier can contain other suitable pharmaceutical additives such as solubilizers, emulsifiers, buffers, suspending agents, thickening agents, viscosity regulators, stabilizers or osmo-regulators. Suitable examples of liquid carriers for oral and parenteral administration include water (particularly containing additives as above, e.g., cellulose derivatives, preferably sodium carboxymethyl cellulose solution), alcohols (including monohydric alcohols and polyhydric alcohols, e.g., glycols) and their derivatives, and oils (e.g., fractionated coconut oil, arachis oil, corn oil, peanut oil, and sesame oil). For parenteral administration, the carrier can also be an oily ester such as ethyl oleate and isopropyl myristate. Sterile liquid carriers are used in sterile liquid form for parenteral administration.

Optionally, additives customarily employed in the preparation of pharmaceutical compositions may be included in the compositions. Such components include, e.g., sweeteners or other flavoring agents, coloring agents, preservatives, and antioxidants, e.g., vitamin E, ascorbic acid, butylated hydroxytoluene (BHT) and butylated hydroxyanisole (BHA).

Liquid pharmaceutical compositions that are sterile solutions or suspensions can be utilized by, for example, intramuscular, intraperitoneal or subcutaneous injection. Sterile solutions can also be administered intravenously. Oral administration may be either liquid or solid composition form.

Suitably, when prepared for use as an inhalant, the pharmaceutical compositions are prepared as fluid unit doses using a compound of formulae (I) and (II) and a suitable pharmaceutical vehicle for delivery by an atomizing spray pump, or by dry powder for insufflation. For use as aerosols, the compound is formulated for and packaged in a pressurized aerosol container together with a gaseous or liquefied propellant, for example, dichlorodifluoromethane, carbon dioxide, nitrogen, propane, and the like, with the usual components such as cosolvents and wetting agents, as may be necessary or desirable. For example, also provided is the delivery of a metered dose for oral or intranasal inhalation in one, two, or more actuations. Suitably, a dose is delivered in one or two actuations. However, other suitable delivery methods may be readily determined.

Preferably the pharmaceutical composition is in unit dosage form, e.g., as tablets or capsules. In such form, the composition is sub-divided in unit doses containing appropriate quantities of the active ingredient. The unit dosage forms can be packaged compositions, for example, packeted powders, vials, ampoules, prefilled syringes or sachets containing liquids. The unit dosage form can be, for example, a capsule or tablet itself, or it can be the appropriate number of any such compositions in package form.

As described herein a therapeutically or prophylactically useful amount of a compound of formulae (I) and (II) is that amount of a compound which alleviates the symptoms of the disease, e.g., AD, or which prevents the onset of symptoms or the onset of more severe symptoms.

In still another embodiment, these compounds are formulated in pharmaceutical compositions useful in treatment of cancers associated with notch processing. A method of treating such cancers using these compounds is provided.

The useful amounts of a compound may vary depending upon the formulation and route of delivery. For example, higher amounts may be delivered orally than when the compound is formulated for injection or inhalation, in order to deliver a biologically equivalent amount of the drug. Suitably, an individual dose (i.e., per unit) of a compound is in the range from about 1 μg/kg to about 10 g/kg. However, in certain embodiments, these doses may be selected from a lower range, e.g., from about 1 μg/kg to about 200 mg/kg, more preferably 10 μg/kg to about 10 mg/kg, and most preferably about 100 μg/kg to about 1 mg/kg. Desirably, these amounts are provided on a daily basis. However, the dosage to be used in the treatment or prevention of a specific cognitive deficit or other condition may be subjectively determined by the attending physician. The variables involved include the specific cognitive deficit and the size, age and response pattern of the subject (used interchangeable with patient). For example, based upon the activity profile and potency of the compounds described herein, a starting dose of about 130 to about 300 mg per day with gradual increase in the daily dose to about 1000 mg per day may provide the desired dosage level in the patient.

Alternatively, the use of sustained delivery devices may be desirable in order to avoid the necessity for the patient to take medications on a daily basis. “Sustained delivery” is defined as delaying the release of an active agent, i.e., a compound of formula I and/or formula II, until after placement in a delivery environment, followed by a sustained release of the agent at a later time. Those of skill in the art know suitable sustained delivery devices. Examples of suitable sustained delivery devices include, e.g., hydrogels (U.S. Pat. Nos. 5,266,325; 4,959,217; and 5,292,515), an osmotic pump, such as described by Alza (U.S. Pat. Nos. 4,295,987 and 5,273,752) or Merck (European Patent No. 314,206), among others; hydrophobic membrane materials, such as ethylenemethacrylate (EMA) and ethylenevinylacetate (EVA); bioresorbable polymer systems (see, e.g., International Patent Publication No. WO 98/44964, Bioxid and Cellomeda; U.S. Pat. No. 5,756,127 and U.S. Pat. No. 5,854,388); other bioresorbable implant devices have been described as being composed of, for example, polyesters, polyanhydrides, or lactic acid/glycolic acid copolymers (see, e.g., U.S. Pat. No. 5,817,343 (Alkermes Inc.)), all of which documents which are hereby incorporated by references. For use in such sustained delivery devices, the compounds may be formulated as described herein.

In another aspect, pharmaceutical kits for delivery of a product are provided. Suitably, the kit contains packaging or a container with the compound formulated for the desired delivery route. For example, if the kit is designed for administration by inhalation, it may contain a suspension containing a compound of formulae (I) and/or (II) formulated for aerosol or spray delivery of a predetermined dose by inhalation. Suitably, the kit contains instructions on dosing and an insert regarding the active agent. Optionally, the kit may further contain instructions for monitoring circulating levels of product and materials for performing such assays including, e.g., reagents, well plates, containers, markers or labels, and the like. Such kits are readily packaged in a manner suitable for treatment of a desired indication. For example, the kit may also contain instructions for use of the spray pump or other delivery device.

Other suitable components to such kits will be readily apparent to one of skill in the art, taking into consideration the desired indication and the delivery route. The doses may be repeated daily, weekly, or monthly, for a predetermined length of time or as prescribed.

In yet another embodiment, one or more of the compounds of the invention can be used to monitor therapy with a gamma secretase inhibitor drug having a structure, e.g., of the heterocyclic sulfonamide compounds such as those described in U.S. Pat. No. 6,878,742 and U.S. Pat. No. 6,610,734 and fluoro- and trifluoroalkyl-containing heterocyclic sulfonamide compounds such as those described in U.S. Pat. No. 7,300,951 and US-2007-0254929-A1, which are incorporated by reference. Still other drugs include the phenylsulfonamides described in U.S. Pat. No. 7,166,622, or the drugs described in US-2005-0171180, which are incorporated by reference.

For example, a compound of the invention can be used to monitor therapy with a compound of the structure:

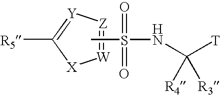

wherein:

T is selected from the group consisting of CHO, COR_{8″}, and C(OH)R_{1″}R_{2″}; R_{1″} and R_{2″} are independently selected from the group consisting of hydrogen, lower alkyl, substituted lower alkyl (an alkyl chain of 1-6 carbon atoms in length, preferably 1-4 carbon atoms, wherein substituted is defined as above for substituted alkyl), CF₃, alkenyl, substituted alkenyl, alkynyl, and substituted alkynyl; R_{3″} is selected from the group consisting of hydrogen, lower alkyl and substituted lower alkyl; R_{4″} is selected from the group consisting of (CF₃)ₙalkyl, (CF₃)ₙ(substituted alkyl), (CF₃)ₙalkylphenyl, (CF₃)ₙalkyl(substituted phenyl), and (F)ₙcycloalkyl; n=1-3; R_{5″} is selected from the group consisting of hydrogen, halogen, CF₃, diene fused to Y when Y═C, and substituted diene fused to Y when Y═C; W, Y and Z are independently selected from the group consisting of C, CR_{6′} and N with the proviso that at least one of W or Y or Z must be C; R_{6″} is selected from the group consisting of hydrogen, halogen, lower alkyl, and substituted lower alkyl; X is selected from the group consisting of O, S, SO₂, and NR_{7″}; R_{7″} is selected from the group consisting of hydrogen, lower alkyl, substituted lower alkyl, benzyl, substituted benzyl, phenyl, and substituted phenyl; and R_{8″} is selected from the group consisting of lower alkyl, CF₃, phenyl, and substituted phenyl; or a pharmaceutically acceptable salt, hydrate, or prodrug thereof.

In one embodiment, the compound to be monitored is 5-Chloro-thiophene-2-sulfonic acid [(1 S,2R)-2-(3,5-difluoro-phenyl)-3,3,3-trifluoro-1-hydroxymethyl-propyl]-amide. In one embodiment, this compound has the structure:

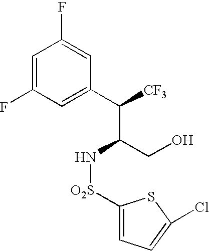

In another embodiment, the compound is 5′-chloro-N-[3,3,3-trifluoro-2-(trifluoromethyl)-1-S-(hydroxymethyl)propyl]thiophene-2′-sulfonamide. In one embodiment, this compound has the structure:

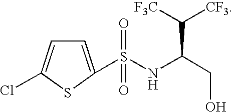

When used as a reagent and/or a standard, the compounds may be labeled, e.g., with a radioactive, fluorescent, or calorimetric tag. One or more of these compounds can serve as a standard, i.e., for comparison purposes, in a method for detecting the presence of a metabolite in a sample. A “sample” as used herein refers to a biological sample, such as, for example, tissue or fluid isolated from an individual (including without limitation plasma, serum, cerebrospinal fluid, urine, lymph, tears, saliva and tissue sections) or from in vitro cell culture constituents, as well as samples from the environment.

In another embodiment, one or more of the metabolites can be used to generate an antibody or antibodies that are used to detect the presence of the GSI metabolites in a sample. Suitably, the antibody is a monoclonal or polyclonal antibody specific for the compound. In one desirable embodiment, such an antibody selectively binds to the compound, and distinguishes that metabolite from the parent compound and other metabolites thereof.

The term “antibody” as used herein is intended to include fragments thereof which are specifically reactive with tanaproget and/or its metabolites, e.g., an Fv fragment and a F(ab)₂ fragment.

An antibody specific to a compound as described herein can be prepared using standard techniques wherein the antigen is a derivative of the invention. See, e.g., Sambrook, Molecular Cloning: A Laboratory Manual, Cold Spring Harbor Press, Cold Spring Harbor, N.Y. The polyclonal and monoclonal antibodies to specific sites of a compound as described herein may be used for development of immunoassays or therapeutic drug monitoring (TDM) kits. Such assays could include, but are not limited to, direct, inhibition, competitive or sandwich immunoassays (ELISA or other assay systems), RIA, solid or liquid phase assays or automated assay systems.

Where a competitive assay is used, the competitor for the antibody may be a compound as described herein bound to the assay plate, or a labeled derivative, e.g., a fluorolabeled derivative, a radiolabeled derivative, or a tritiated derivative. Where desired, a kit can be used to facilitate the methods of the invention. A kit of the invention may contain an appropriately labeled tracer, an antibody, standard, instructions for use, and packaging. The label for the tracer may be any suitable label, e.g., a radioactive, fluorescent or calorimetric label. Where convenient, the components of the kit may be in lyophilized form.

The assay procedure of the invention has the advantages that it may be carried out rapidly and simply using standard bioanalytical equipment to give accurate and reproducible results. Also, whole blood may be used without the need for extraction. In one embodiment, an assay kit suitable for detecting the amount of the metabolite in a sample (e.g., blood or urine) is provided. In one embodiment, the kit comprises a binding competitor that displaces the pharmaceutical from the metabolite in the sample; and an antibody that binds to the pharmaceutical but not significantly to the binding competitor.

The following examples are provided to illustrate the production of representative compounds of the invention. One skilled in the art will appreciate that although specific reagents and conditions are outlined in the following examples, these reagents and conditions are not a limitation on the present invention.

### Example 1

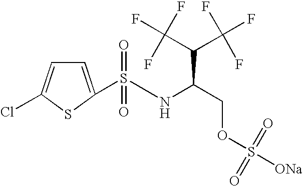

### (2S)-2-{[(5-chloro-2-thienyl)sulfonyl]amino}-4,4,4-trifluoro-3-(trifluoromethyl)butyl Hydrogen Sulfate, Sodium Salt

Chlorosulfonic acid (0.060 mL, 0.90 mmol) was added dropwise to a stirring solution of 5-chloro-N-[(1S)-3,3,3-trifluoro-1-(hydroxymethyl)-2-(trifluoromethyl)propyl]thiophene-2-sulfonamide) (0.175 g, 0.45 mmol) in ethyl acetate (1.4 mL) at 0° C. under nitrogen over a period of 5 min. The mixture was then stirred at 0° C. and monitored by thin layer chromatography (TLC) and nuclear magnetic resonance (NMR). The reaction was complete in 30 min. Saturated aqueous sodium bicarbonate (0.30 g, 3.60 mmol in 1.4 mL of water) was added carefully and slowly to the reaction mixture at 0° C. and the mixture was stirred at 0° C. for 30 min. and then at room temperature for 30 min. The reaction mixture was diluted with ethyl acetate (80 mL). The ethyl acetate mixture was dried with anhydrous sodium sulfate (oven dried). The solid was removed by filtration. The filtrate was evaporated to afford a solid. This solid was recrystallized from methylene chloride and pentane (8:2) to give the title compound as a white solid (0.090 g).

Mass spectrum (ES, [M-H]−) m/z 469.9.

High resolution mass spectrometry (HRMS): calcd for C₉H₈ClF₆NO₆S₃, [M-H]−, 469.9028; found (ESI, [M-H]−), 469.9029

### Example 2

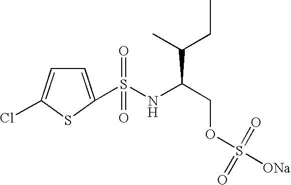

### (2S,3R)-2-{[(5-chloro-2-thienyl)sulfonyl]amino}-3-methylpentyl Hydrogen Sulfate, Sodium Salt

Chlorosulfonic acid (0.034 mL, 0.50 mmol) was added dropwise to a stirring solution of 5-chloro-N-[(1S,2S)-1-(hydroxymethyl)-2-methylbutyl]-2-thiophenesulfonamide (0.34 mmol) in ethyl acetate (0.8 mL) at 0° C. under nitrogen over a period of 5 min. The mixture was then stirred at 0° C. and monitored by TLC and NMR. The reaction was complete in 30 min. Sodium bicarbonate (0.170 g, 2.0 mmol) was added to the reaction mixture. After the mixture was stirred at 0° C. for 10 min., water (0.75 mL) was added slowly to the mixture at 0° C. The reaction mixture was stirred at room temperature for 15 min and diluted with ethyl acetate (40 mL). The ethyl acetate mixture was dried with anhydrous sodium sulfate (oven dried). Filtration and evaporation gave the title compound as a white solid (0.063 g).

Mass spectrum (ES, [M-H]−) m/z 376.0

HRMS: calcd for C₁₀H₁₆ClNO₆S₃, [M-H]−, 375.9750; found (ESI, [M-H]−), 375.9751.

### Example 3

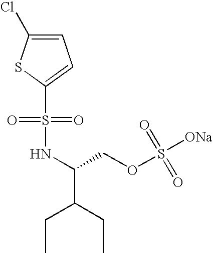

### (2S)-2-{[(5-chloro-2-thienyl)sulfonyl]amino}-3-ethylpentyl Hydrogen Sulfate, Sodium Salt

The title compound was prepared from 5-chloro-N-[(1S)-2-ethyl-1-(hydroxymethyl)butyl]-2-thiophenesulfonamide in substantially the same manner as described in Example 1. The product was obtained as white solid.

Mass spectrum (ES, [M-H]−) m/z 390.0

HRMS: calcd for C₁₁H₁₈CINO₆S₃, [M-H]−, 389.9907; found (ESI, [M-H]−), 389.9919.

### Example 4

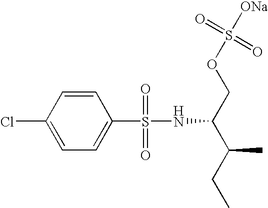

### (2S,3S)-2-{[(4-chlorophenyl)sulfonyl]amino}-3-methylpentyl Hydrogen Sulfate, Sodium Salt

The title compound was prepared from 4-chloro-N-((1S,2S)-1-hydroxymethyl-2-methyl-butyl)-benzenesulfonamide in substantially the same manner as described in Example 2. The product was obtained as white solid.

Mass spectrum (ES, [M-H]−) m/z 370.1

HRMS: calcd for C₁₂H₁₈ClNO₆S₂, [M-H]−, 370.0186; found (ESI, [M-H]−), 370.0197.

### Example 5

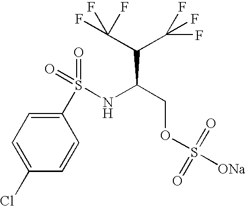

### ((2S)-2-{[(4-chlorophenyl)sulfonyl]amino}-4,4,4-trifluoro-3-(trifluoromethyl)butyl Hydrogen Sulfate, Sodium Salt

The title compound was prepared from 4-chloro-N-[(1S)-3,3,3-trifluoro-1-(hydroxymethyl)-2-(trifluoromethyl)propyl]benzenesulfonamide in substantially the same manner as described in Example 2. The product was obtained as white solid.

Mass spectrum (ES, [M-H]−) m/z 464

HRMS: calcd for C₁₁H₁₀ClF₆NO₆S₂, [M-H]−, 463.9464; found (ESI, [M-H]−), 463.9473.

### Example 6

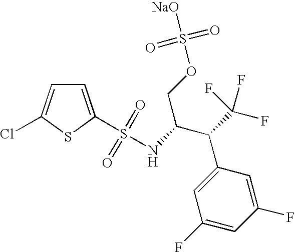

### (2S,3R)-2-{[(5-chloro-2-thienyl)sulfonyl]amino}-3-(3,5-difluorophenyl)-4,4,4-trifluorobutyl Hydrogen Sulfate, Sodium Salt

The title compound was prepared from 5-chloro-N-[(1S,2R)-2-(3,5-difluorophenyl)-3,3,3-trifluoro-1-(hydroxymethyl)propyl]thiophene-2-sulfonamide in substantially the same manner as described in Example 2. The product was obtained as white solid.

Mass spectrum (ES, [M-H]−) m/z 514

HRMS: calcd for C₁₄H₁₁ClF₅NO₆S₃, [M+Na]+, 537.9253; found (ESI, [M+Na]+), 537.9249.

### Example 7

### Pharmacology

A. Beta Amyloid Activity

The activity of compounds in lowering the generation of Aβ40 and Aβ42, the most abundant forms of beta amyloid, from APP (amyloid precursor protein) was measured in an electrochemiluminescent (ECL) assay of conditioned media from chinese hamster ovary (CHO) cells stably expressing the human amyloid precursor protein (APP) reporter construct, hAPP-REPNL₇₅₁ [Sughir, R. et al.; J. Biolog. Chem. (1992) 267: 25602-25608]. Aβ peptides were secreted at high levels into cell culture media by cells carrying the human APP transgene; compounds were tested for their capacity to modulate this production. The Aβ peptide in the conditioned medium was quantitated by a sandwich immunoassay with the Meso Scale Discovery® (MSD®) ECL detection system. Cell metabolism was measured using MTS salt kit (containing a tetrazolium compound 3-(4,5-dimethylthiazol-2-yl)-5-(3-carboxymethoxyphenyl)-2-(4-sulfophenyl)-2H-tetrazolium, inner salt; MTS(a) which measures the mitochondrial activity of cells by the bioreduction of Owen's reagent). This assay consists of the following protocol:
1. Aβ-Lowering Assay.
   a. hAPP-REPNL₇₅₁ CHO cells were seeded into 96 well plates and incubated until about 60-70% confluence.
   b. Medium was removed, cells were washed, and fresh serum-free medium (Ultraculture) was put on cells.
   c. Compounds were diluted and then added to the cell medium.
   d. Cells were incubated, with compounds, for indicated times.
   e. Streptavidin coated Meso Scale Discover (MSD) plates (MSD standard Multi-Array™ 96 plate, cat. #P11SA-1) were washed 3× with TTBS (Tris-buffered saline, the Tween® 20 reagent).
   f. 20 μL conditioned medium were removed from the cells, and added to the TTBS prewashed MSD® plates.
   g. Standard curve dilutions of synthetic Aβ40 and Aβ42 were prepared and added to the MSD® plates.
   h. A reagent mix was prepared in 1% MSD® Blocker A (appropriate concentration of biotinylated 6E10 antibody, detection antibodies to Aβ40, Aβ42, and MSD ruthinylated tag antibody).
   i. 20 μL reagent mix was distributed to sample plate.
   j. Plates were incubated with shaking overnight at 4° C.
   k. Plates were washed 3× with TTBS.
   l. 150 μL read buffer was added per well (MSD Read Buffer T, cat#R92TC-2, made 1× with distilled water).
   m. Plates were read in MSD plate reader within 2 hours.
2. MTS Assay:
   a. Cells were seeded into 96 well plates and incubated until about 60-70% confluence.
   b. Medium was removed, cells were washed, and fresh serum-free medium (Ultraculture) was put on cells.
   c. Compounds were diluted and then added to the cell medium.
   d. Cells were incubated, with compounds, for indicated times.
   e. Using a robot, conditioned medium was removed from the cells, and transferred to a TTBS prewashed MSD plate.
   f. Cells were washed 2× in phosphate buffered saline (PBS) and an MTS solution was plated onto the cells. After 1 hour, they were read on a plate reader at 560 nm to determine metabolic activity.
3. Analysis of Results.
   Assays were accepted or rejected based upon specific performance criteria, including regression coefficient of standard curve, adequate signal to noise ratio, sample signals lying within the range of the standard curve, etc. The specific parameters were established for each tissue type prior to performing an assay, and were included in the full analytical procedure.
   Plate data from both assays (MTS and MSD ELISA) were transferred into a Microsoft Excel® spreadsheet to determine the toxicity, and inhibition caused by the compounds. Standard curves of Aβ were generated using LSW toolbar Hill slope model 42, with 1/y weighting (general sigmoidal curve with Hill slope, a to d; y=(a−d)/(1+(x/c)̂b)+d). Inhibition data were expressed as a percent of the average of the Aβ values in the vehicle treated wells (all values were background subtracted), using LSW toolbar Hill Slope model 68 (ligand-receptor binding/sigmoidal, with Hill slope and Bmax to 0; y=Bmax*(1−(x̂n/(K̂n)+x̂n)), after raw values were translated/transformed into absolute Aβ values utilizing the standard curve generated as described above (e.g., back calculate absolute Aβ values from the raw values utilizing standard curve). Onboard controls to verify assay performance were checked to assure that amyloid is within linear detection range of the assay, that cells were expressing correctly, and that the MSD itself was performing according to quality control (QC) standards.
4. Response Determination.
   % Inhibition >50% may be considered a positive response or an interesting result in this assay; EC₅₀ determination are determined.

The reference standards used for the beta amyloid assay were:
COMPOUNDS: 5-chloro-N-[(1S,2R)-2-ethyl-4,4,4-trifluoro-1-(hydroxymethyl)butyl]thiophene-2-sulfonamide (EC₅₀αβ40=6 nM, EC₅₀αβ42=5 nM)
(2S)-2-hydroxy-3-methyl-N-((1S)-1-methyl-2-{[(1S)-3-methyl-2-oxo-2,3,4,5-tetrahydro-1H-3-benzazepin-1-yl]amino}-2-oxoethyl)butanamide (EC₅₀αβ40=109 nM, EC₅₀αβ42=79 nM)

B. Functional Notch Assay

The activity of compounds in cleaving Notch can be measured in a whole cell functional Notch assay. This is an assay to determine the effects of compounds on the S3 (γ-secretase like) processing of Notch. The assay permits measurement of the inhibition of S3 cleavage activity as revealed by reduced transactivation of a reporter gene: specifically, a constitutively active form of Notch (having the extracellular domain deletion) when cleaved by γ-secretase releases the Notch intracellular domain (NICD) which transactivates the soluble alkaline phosphatase (SEAP) gene driven by the HES promoter. SEAP transactivation is then detected by luminescent assay. The assay consists of the following protocol:
1. Materials and Methods:
   (a) Materials: The P8H6 inducible stable cell line was derived from T-REx™ CHO-K1, stably expressing Tet repressor, Notch (pZX1) and HES1-SEAP (pZX2) reporter constructs. A Wallace 1450® Victor Luminescence Counter is used to measure luminescent signal. All other materials and reagents were of highest quality available and were all from commercial sources.
(b) Construct Description:
   pZX1: Mouse Notch1 with deleted extracellular domain cloned at Hind III/Xho I sites of pcDNA5/TO vector: Ampicillin Resistance for bacteria, hygromycin resistance for cells.
   pZX2: Mouse HES1 promoter together with reporter gene SEAP cloned at Not I/Apa I sites of pcDNA3.1 with deletion of CMV promoter.
(c) Procedure:
   P8H6 cells were grown in Ham's F12 complete growth media containing 10% fetal bovine serum ((FBS), with reduced tetracycline), 2 mM of L-glutamine, 1 mg/mL of hygromycin, 1 mg/mL of the Geneticin® antibiotic, 10 μg/mL of Blasticidin and 1% Penicillin-Streptomycin were supplemented with doxycline with final induction concentration of 0.2 μg/mL. The cells were seeded in 96-well tissue culture plate at 8000/well.
   Test compounds in 0.2% dimethylsulfoxide (DMSO)/25 mM HEPES (4-(2-hydroxyethyl)-1-piperazineethanesulfonic acid) buffer were diluted at final working concentration of 20 μM, 6.7 μM, 2.2 μM, 741 nM, 247 nM, 82 nM, 27 nM, 9 nM and 3 nM. Diluted compounds then were added to P8H6 cells in 96-well tissue culture plates. For vehicle samples, the same volume of 0.2% DMSO/25 mM HEPES buffer was added.
   Cells were incubated at 37° C., 5% CO₂ for 48 hours.
   SEAP levels in the conditioned media were assessed using the Clontech GreatEscAPe™ SEAP Chemiluminescence detection kit according to the manufacturer's instruction. Briefly, 15 μL of conditioned media were mixed with 45 μL of the dilution buffer and incubated at 65° C. for 45 min. After cooling, assay buffer was added, and the sample was incubated with the CSPD® substrate.
   Luminescence was measured in a Wallace 1450 Victor luminescence counter.
2. Analysis of Results:
   EC₅₀ was the compound concentration that was estimated to provide a 50% reduction of maximum response in Notch induced secreted alkaline phosphatase (SEAP) levels. An EC₅₀ from a given assay may only be used for averaging if it has met the following criteria:
   (a) There is a lower dose in the assay which gives a % Notch Activity >60% (bracketing lower dose).
   (b) There is a higher dose in the assay which gives a % Notch Activity <30% (bracketing higher dose), and the maximum inhibition is reached (e.g., a given compound might achieve 80% inhibition (% Notch activity is ˜20%) over several doses, and therefore 20% would be considered the maximum inhibition).
      Data were analyzed in Microsoft® Excel format. EC₅₀ was calculated by line spin wave theory (LSW) using sigmoidal inhibition from B0 to nsb model (model 59). Each assay plate contained a dose response curve for the reference compound 5-chloro-N-[(1S,2R)-2-ethyl-4,4,4-trifluoro-1-(hydroxymethyl)butyl]thiophene-2-sulfonamide. This compound is discussed in International Patent Publication No. WO 2004/092155. The following data is not shown in that publication, but is provided below. The EC₅₀ for this reference compound must fall within the range of 150-350 nM for the assay plate to be accepted.
3. Notch assay reference compounds:
   5-chloro-N-[(1S,2R)-2-ethyl-4,4,4-trifluoro-1-(hydroxymethyl)butyl]thiophene-2-sulfonamide (EC₅₀=225 nM).
   (2S)-2-hydroxy-3-methyl-N-((1S)-1-methyl-2-{[(1S)-3-methyl-2-oxo-2,3,4,5-tetrahydro-1H-3-benzazepin-1-yl]amino}-2-oxoethyl)butanamide (EC₅₀=68 nM). This compound is discussed in International Patent Publication No. WO 2002/47671, but this data is not shown.
   The beta amyloid inhibitory activity of compounds of formula (I) and (II) was determined using the MSD ECL assay. Inhibition of Notch processing was measured using the stable transfection reporter assay. See, Table 1 below.

TABLE 1
Compounds and biological data

| | C40* | C42* | N9319A^{⊖} | |
|---|---|---|---|---|
| Ex # | nM | nM | nM | Name |
| 1 | 447 | 445 | 3005 | (2S)-2-{[(5-chloro-2-thienyl)sulfonyl]amino}- |
| | | | | 4,4,4-trifluoro-3-(trifluoromethyl)butyl sodium |
| | | | | sulfate |
| 2 | 15000 | 15000 | NT | (2S,3R)-2-{[(5-chloro-2-thienyl)sulfonyl]amino}- |
| | | | | 3-methylpentyl sodium sulfate |
| 3 | 243 | 234 | NT | (2S)-2-{[(5-chloro-2-thienyl)sulfonyl]amino}-3- |
| | | | | ethylpentyl sodium Sulfate |
| 4 | 15000 | 15000 | NT | (2S,3S)-2-{[(4-chlorophenyl)sulfonyl]amino}-3- |
| | | | | methylpentyl sodium sulfate |
| 5 | 4028 | 4147 | NT | (2S)-2-{[(4-chlorophenyl)sulfonyl]amino}-4,4,4- |
| | | | | trifluoro-3-(trifluoromethyl)butyl sodium sulfate |
| 6 | 1742 | 1288 | NT | (2S,3R)-2-{[(5-chloro-2-thienyl)sulfonyl]amino}- |
| | | | | 3-(3,5-difluorophenyl)-4,4,4-trifluorobutyl |
| | | | | sodium sulfate |

*EC₅₀ For inhibition of Aβ40/Aβ42 in nM as determined by electrochemiluminescent assay
^{⊖}ED₅₀ For inhibition of Notch in nM.
NT: Not tested

All publications cited in this specification are incorporated herein by reference. While the invention has been described with reference to particular embodiments, it will be appreciated that modifications can be made without departing from the spirit of the invention. Such modifications are intended to fall within the scope of the appended claims.

1. A composition comprising a pharmaceutically acceptable carrier and a compound having the structure of formula I or II or a pharmaceutically acceptable salt and/or hydrate thereof: 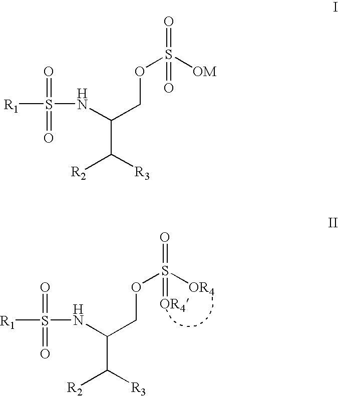
wherein:
R₁ is substituted aryl or substituted heteroaryl;
R₂ and R₃ are independently selected from the group consisting of CF₃, substituted phenyl, C₁-C₄ alkyl, substituted C₁-C₄ alkyl, (CF₃)ₙC₁-C₄ alkyl, and (CF₃)ₙ(substituted C₁-C₄ alkyl), wherein n is 1, 2 or 3; provided that when R₂ or R₃ is CF₃, the other is not an unsubstituted alkyl; and
R₄ and R_{4′} are independently selected from the group consisting of M, C₁-C₄ alkyl, phenyl, and benzyl, wherein M is a metal ion selected from the group consisting of sodium, lithium, calcium, magnesium and potassium; Or
R₄ and R_{4′} are taken together to form a cyclic structure selected from the group consisting of:

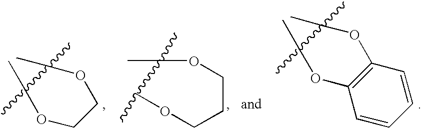

2. The composition according to claim 1, wherein R₁ is a substituted phenyl, substituted pyridyl, or a substituted thienyl.

3. The composition according to claim 2, wherein R₁ is substituted phenyl or substituted thienyl, which are substituted with a chloro or a fluoro group.

4. The composition according to claim 1, wherein R₁ has S-stereochemistry at the carbon bearing the sulfonamide nitrogen atom.

5. The composition according to claim 1, wherein R₂ is selected from the group consisting of CF₃ and C₁-C₃ alkyl.

6. The composition according to claim 1, wherein R₃ is selected from the group consisting of CF₃, C₁-C₃ alkyl, and difluorophenyl.

7. The composition according to claim 1, wherein the compound has formula I and M is sodium.

8. The composition according to claim 1, wherein the compound is selected from the group consisting of (2S)-2-{[(5-chloro-2-thienyl)sulfonyl]amino}-4,4,4-trifluoro-3-(trifluoromethyl)butyl sodium sulfate;
(2S,3R)-2-{[(5-chloro-2-thienyl)sulfonyl]amino}-3-methylpentyl sodium sulfate;
(2S)-2-{[(5-chloro-2-thienyl)sulfonyl]amino}-3-ethylpentyl sodium sulfate;
(2S,3S)-2-{[(4-chlorophenyl)sulfonyl]amino}-3-methylpentyl sodium sulfate;
(2S)-2-{[(4-chlorophenyl)sulfonyl]amino}-4,4,4-trifluoro-3-(trifluoromethyl)butyl sodium sulfate; and
(2S,3R)-2-{[(5-chloro-2-thienyl)sulfonyl]amino}-3-(3,5-difluorophenyl)-4,4,4-trifluorobutyl sodium sulfate.

9. The composition according to claim 1, wherein said composition is formulated for intravenous delivery.

10. A synthetically-produced compound having the structure of formula I or II: 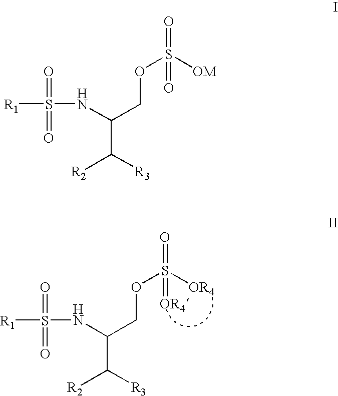
wherein:
R₁ is substituted aryl or substituted heteroaryl;
R₂ and R₃ are independently selected from the group consisting of CF₃, substituted phenyl, C₁-C₄ alkyl, substituted C₁-C₄ alkyl, (CF₃)ₙC₁-C₄ alkyl, and (CF₃)ₙ(substituted C₁-C₄ alkyl), wherein n is 1, 2 or 3; provided that when R₂ or R₃ is CF₃, the other is not an unsubstituted alkyl; and
R₄ and R_{4′} are independently selected from the group consisting of M, C₁-C₄ alkyl, phenyl, and benzyl, wherein M is a metal ion selected from the group consisting of sodium, lithium, calcium, magnesium and potassium; Or
R₄ and R_{4′} are taken together to form a cyclic structure selected from the group consisting of:

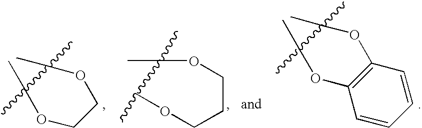

11. The compound according to claim 10, wherein R₁ is a substituted phenyl, substituted pyridyl, or a substituted thienyl.

12. The compound according to claim 11, wherein R₁ is substituted phenyl or substituted thienyl, wherein the substituent is a chloro or fluoro group.

13. The compound according to any of claims 10 to 12, wherein R₁ has S-stereochemistry at the carbon bearing the sulfonamide nitrogen atom.

14. The compound according to claim 10, wherein R₂ is selected from the group consisting of CF₃ and C₁-C₃ alkyl.

15. The compound according to claim 10, wherein R₃ is selected from the group consisting of CF₃, C₁-C₃ alkyl, and difluorophenyl.

16. The compound according to claim 10, wherein the compound has formula I and M is sodium.

17. The compound according to claim 10, wherein the compound is selected from the group consisting of:
(2S)-2-{[(5-chloro-2-thienyl)sulfonyl]amino}-4,4,4-trifluoro-3-(trifluoromethyl)butyl sodium sulfate;
(2S,3R)-2-{[(5-chloro-2-thienyl)sulfonyl]amino}-3-methylpentyl sodium sulfate;
(2S)-2-{[(5-chloro-2-thienyl)sulfonyl]amino}-3-ethylpentyl sodium sulfate;
(2S,3S)-2-{[(4-chlorophenyl)sulfonyl]amino}-3-methylpentyl sodium sulfate;
(2S)-2-{[(4-chlorophenyl)sulfonyl]amino}-4,4,4-trifluoro-3-(trifluoromethyl)butyl sodium sulfate; and
(2S,3R)-2-{[(5-chloro-2-thienyl)sulfonyl]amino}-3-(3,5-difluorophenyl)-4,4,4-trifluorobutyl sodium sulfate.

18. A method of increasing the circulating half-life of a compound useful in lowering beta amyloid levels in a subject, said method comprising administering to a subject a compound having the structure of formula I or II or a pharmaceutically acceptable salt and/or hydrate thereof: 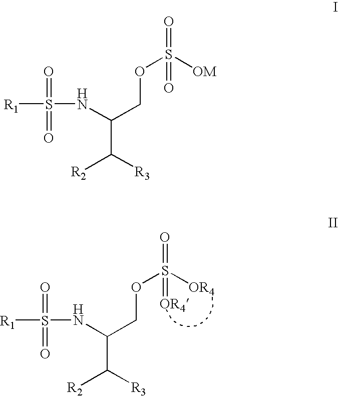
wherein:
R₁ is substituted aryl or substituted heteroaryl;
R₂ and R₃ are independently selected from the group consisting of CF₃, substituted phenyl, C₁-C₄ alkyl, substituted C₁-C₄ alkyl, (CF₃)ₙC₁-C₄ alkyl, and (CF₃)ₙ(substituted C₁-C₄ alkyl), wherein n is 1, 2 or 3; provided when that R₂ or R₃ is CF₃, the other is not an unsubstituted alkyl; and
R₄ and R_{4′} are independently selected from the group consisting of M, a C₁-C₄ alkyl, a phenyl, and a benzyl, wherein M is a metal ion selected from the group consisting of sodium, lithium, calcium, magnesium and potassium; Or
R₄ and R_{4′} are taken together to form a cyclic structure selected from the group consisting of:

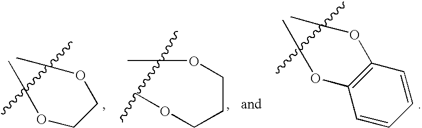

19. The method according to claim 18, wherein the compound useful in lowering beta amyloid levels in a subject is selected from the group consisting of 5-Chloro-thiophene-2-sulfonic acid [2-(3,5-difluoro-phenyl)-3,3,3-trifluoro-1-hydroxymethyl-propyl]-amide and 5′-chloro-N-[3,3,3-trifluoro-2-(trifluoromethyl)-1-S-(hydroxymethyl)propyl]thiophene-2′-sulfonamide.

20. A method of delivering 5-Chloro-thiophene-2-sulfonic acid [(1S,2R)-2-(3,5-difluoro-phenyl)-3,3,3-trifluoro-1-hydroxymethyl-propyl]-amide to a subject, said method comprising administering to a subject a prodrug thereof in a pharmaceutical composition, wherein said prodrug has the structure:

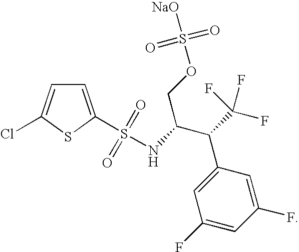

21. A method of treating cancer, said method comprising administering to a subject a composition according to claim 1.

22. The method according to claim 21, wherein the composition is administered intravenously.

23. A method of preparing a compound of formula I, said method comprising:
(a) reacting a sulfonamide alcohol of formula III 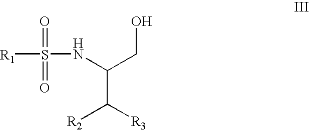
with a chlorosulfonic acid;
(b) treating the product of (a) with a base to afford a sulfate of formula (I): 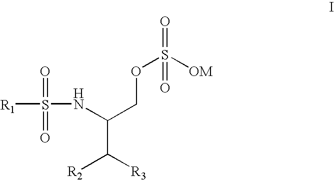
wherein:
R₁ is substituted aryl or substituted heteroaryl;
R₂ and R₃ are independently selected from the group consisting of CF₃, substituted phenyl, C₁-C₄ alkyl, substituted C₁-C₄ alkyl, (CF₃)ₙC₁-C₄alkyl, and (CF₃)ₙ(substituted C₁-C₄ alkyl), wherein n is 1, 2 or 3; provided that when R₂ or R₃ is CF₃, the other is not an unsubstituted alkyl;
R₄ and R_{4′} are independently selected from the group consisting of M, C₁-C₄ alkyl, phenyl, and benzyl, wherein M is a metal ion is selected from the group consisting of sodium, lithium, calcium, magnesium and potassium; Or
R₄ and R_{4′} are taken together to form a cyclic structure selected from the group consisting of:

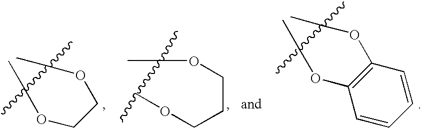

24. The method according to claim 23, wherein step (a) comprises the solvent ethyl acetate.

25. The method according to claim 23, wherein the base is sodium bicarbonate.

26. A method of preparing a compound of formula II, said method comprising:
(a) reacting a sulfonamide alcohol of formula III 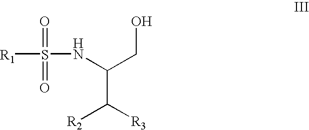
with a chlorophosphonate of the formula (V) 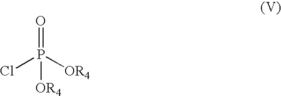
in the presence of a base to afford a phosphonate compound of formula (II) 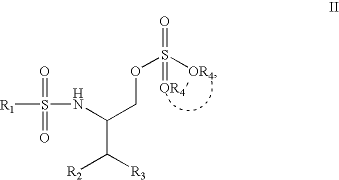
wherein
R₁ is substituted aryl or substituted heteroaryl;
R₂ and R₃ are independently selected from the group consisting of CF₃, substituted phenyl, C₁-C₄ alkyl, substituted C₁-C₄ alkyl, (CF₃)ₙC₁-C₄alkyl, and (CF₃)ₙ(substituted C₁-C₄ alkyl), wherein n is 1, 2, or 3; provided that when R₂ or R₃ is CF₃, the other is not an unsubstituted alkyl;
R₄ and R_{4′} are independently selected from the group consisting of M, C₁-C₄ alkyl, and phenyl, wherein M is a metal ion selected from the group consisting of from sodium, lithium, calcium, magnesium and potassium; Or
R₄ and R_{4′} are taken together to form a cyclic structure selected from the group consisting of: [chemical structure]
